(19) United States
(12) Patent Application Publication
(10) Pub. No.: US 2009/0197251 A1
(43) Pub. Date: Aug. 6, 2009

(54) Process for marking products with nucleic acids for proving the identity and origin of the products

(76) Inventors: David Melchior, Bergisch Gladbach (DE); Bert Poepping, Hamburg (DE)

Correspondence Address: FOX ROTHSCHILD LLP, 2000 MARKET STREET, PHILADELPHIA, PA, 19103, US

(21) Appl. No.: 11/810,577

(22) Filed: Jun. 6, 2007

(30) Foreign Application Priority Data
Jun. 10, 2006 (EP) 06011991.4

(51) Int. Cl. C12Q 1/68

(52) U.S. Cl. 435/6

(57) ABSTRACT

The invention relates generally to product identification and, more particularly, to processes for marking products with nucleic acids to establish the identity and origin of the products, to track them and to determine their quantity in the event of blending or dilution with other products. The invention also relates to the use of nucleic acids which code characters and character sequences for marking products and to products containing nucleic acids which code characters and character sequences.

This invention relates generally to product identification and, more particularly, to processes for marking products with nucleic acids to establish the identity and origin of the products, to track the products and to determine their quantity in the event of blending or dilution with other products. The present invention also relates to the use of nucleic acids which code characters and character sequences for marking products and to products containing nucleic acids which code characters and character sequences.

Normally, considerable difficulties are involved in distinguishing a product made by one manufacturer from the same product made by another manufacturer. This is particularly the case when the manufacturers use the same production process. At the commercial level, this leads to major problems of product falsification, unauthorized distribution and unlicensed sale of a product (for example black marketing and parallel trading). Also associated with this is the problem of false product liability of the manufacturer for products which, although not made and marketed by that manufacturer, cannot be distinguished from the manufacturer's own products in the event of third parties suffering damage through the falsified and possibly inferior products.

All over the world, manufacturers provide their products with a distinguishable, characteristic appearance, for example through the packaging of the product, so that the consumer is able to tell their products from those of the other manufacturers. It follows from this that consumers associate the external appearance of a product with particular quality features and, if they are satisfied with these quality features, prefer those products to other competitive products. However, when consumers have grown accustomed to a product and prefer it to the other competitive products, the problem of product falsification becomes more serious. A falsified product is essentially a product which has an appearance or a trade mark so similar to that of the genuine product that consumers also attribute the falsified product to the original manufacturer and buy it accordingly. The material of a falsified product can be the same as or different from the material used for the genuine product. The material of the falsified product is often the same material, but of inferior quality. In many cases, the material of the product of the honest manufacturer is also mixed to an extent with inferior material.

Besides product falsification, product dilution (also known as blending or mixing) is another major problem for honest manufacturers. A product is said to be diluted when a diluent, possibly a diluent of inferior quality, is added to that product. This problem occurs in particular with liquid products, such as oils or oil-based products.

The above-mentioned problems of product falsification or product dilution not only result in economic damage to the manufacturer of the genuine product, they also impair the original product or an inferior product takes the place of the genuine (original) product, so that consumers may suffer damage which results in serious harm to the reputation of the genuine product.

In the context of competition, manufacturers are anxious not only to distinguish their products from those of competitors or product fraudsters in order to prevent the falsification or dilution of their products, but also to allocate an exact time of manufacture (i.e. day or date of manufacture) and place of manufacture to their own products when they are on the market. This is normally done in the form of an article and/or batch number applied to the product. However, since this article and/or batch number is normally applied to the packaging of the product, it can easily be removed from the product, so that the product can then no longer be assigned to the exact time or place of manufacture.

Accordingly, there is a need for a process for marking products in order to be able to distinguish them from other, possibly falsified and/or diluted products of competitors and, at the same time, to assign them at any time to the place or time of manufacture or batch.

Hitherto, the prior art has only proposed processes which enable products to be marked in order to distinguish them from falsified and/or diluted products of competitors.

EP 1 199 371 A2 describes the marking of products with marker ligands (for example hormones, chemicals, etc.) which, in a detection system (for example in a cell), bind to the ligand binding domain of a transcription factor and activate it which leads to a detectable expression of a reporter gene regulated by that transcription factor.

Similarly, EP 0 409 842 B1 describes the marking of products with a marker compound (for example small organic molecules) which is capable of binding to a complementary binding partner (for example an antibody) to form an immunologically detectable binding pair. Detection is carried out, for example, “enzyme-chemically” in an ELISA reaction or by affinity chromatography.

EP 0 327 163 B1 describes the marking of products with an antigen as marker molecule (for example a protein, lipid, nucleic acid, etc.) which can be identified in a specific immunoassay. Detection of the corresponding antigen/antibody reaction is carried out, for example, in an ELISA reaction.

WO 95/06249 describes the marking of products with a hapten which is optionally covalently bonded to a polymeric compound, such as an oligonucleotide, and the immunological detection of the hapten, for example by an antibody.

WO 98/33162 describes the marking of products with markers which may belong to various classes of compounds and the specific detection of the markers according to their characteristics, for example by immunological, physical or chemical processes. Among the examples mentioned in WO 98/33162 are nucleic acids which are added to the products and which can be detected, for example, by SDS-PAGE, HPLC or nucleic acid hybridization with a complementary nucleic acid.

Similarly, WO 87/06383 describes a process for marking products with a marker (for example a nucleic acid or a protein) and for specifically detecting the marker by a complementarily binding partner (for example a hybridizing complementary nucleic acid or antibody).

Although the documents cited above propose the marking of commercial products with macromolecular substances, such as peptides, proteins or nucleic acids, in order to enable the products to be distinguished from externally identical products of competitors which do not contain that macromolecular substance, there is no known process for the falsification-proof marking of products which, at the same time, enables the manufacturer unequivocally to assign his products to the corresponding time or place of manufacture through the marking used, even after the products have come onto the market.

Accordingly, the problem to be solved in the light of the prior art discussed in the foregoing was to provide a process which would allow falsification-proof marking and, at the same time, exact determination of the manufacturing origin of products.

The problem stated above has been solved by the subject of the present patent claims.

The present invention relates to a process for marking a product to prove its identity and origin, the process comprising the step of marking the product with a nucleic acid, characterized in that the nucleic acid has a coding region of which the sequence is associated with a sequence of characters.

More particularly, the present invention relates to the marking of products with nucleic acids, preferably with DNA. The nucleic acids each have a specific nucleotide sequence which codes a character sequence, preferably an alpha-numeric letter and/or digit sequence, the character sequence coded by the nucleotide sequence representing, for example, the product and/or batch number of the product and thus enabling the exact manufacturing origin and/or time of manufacture of the product to be determined. The present invention also relates to the use of nucleic acids which code character sequences for marking products and to products which contain nucleic acids coding character sequences.

The expression “marking of a product” as used in the present specification is understood to mean the association of a marker with a product so that its origin, identity, time of manufacture, place of manufacture, batch and/or shelf life and other technical information about the product can be determined through the marking. In addition, the identification of a marked product can facilitate the following:
i) monitoring the production process or other processes, including mixing processes of constituents of the product, and packaging processes of the product;
ii) product monitoring for security or logistic processes, for example identification of the country of origin of the product for customs purposes and identification of substances regulated by national legislation, such as poisons and narcotics;
iii) detection and monitoring of contamination of the marked product, including the detection of toxic substances, such as pesticides, herbicides, fertilizers, organic contaminants, such as PBT and dioxins, and other chemicals.

The marking of a product also encompasses the association of a product with a certain concentration of the marker, so that the detection of any change in the product by dilution, changes in concentration or the addition of foreign materials is facilitated.

The term “product” as used in the present specification is understood to encompass any type of product which it is desired to mark for the purpose of identification, proof of origin and/or tracking. For example, such marking of a product may be necessary for detecting unlicensed or illegal copying of the product or for enabling the identification of a falsified and possibly inferior or harmful product in the interests of consumer protection. The product may be present in any physical form. For example, the product can be a liquid or a fluid, a solid, a dispersion, an emulsion, a latex or a semisolid matrix. Non-limiting examples of solid products include pharmaceutical formulations, such as tablets, capsules or powders; solid formulations of agrochemicals, such as insecticides, fungicides, fertilizers and seed material; explosives; textiles, such as clothing; data carriers, such as DVDs, CDs, cassettes, floppy disks; electrical goods, such as televisions, computers and radios; parts of vehicles and cameras; paper, such as documents, confidential papers, advertisements; packaging materials; chemical products, such as dyes, inks, rubber; cosmetic products, such as creams, food products; building materials, such as asphalt additives, roof tiles and concrete. Non-limiting examples of fluids and liquid products include oil-based products, such as lubricating oils, hydraulic oils, lubricants, fuels, such as gasoline, diesel or kerosene, crude petroleum, and petroleum products, paints, paint additives, plastic additives, adhesives, coatings, ceramics, petroleum-based chemicals, such as polymers, perfumes and other cosmetics; beverages, such as water, milk, wine, whiskey, sherry, gin, vodka and other alcoholic and non-alcoholic beverages, liquid pharmaceutical formulations, such as syrups, emulsions and suspensions, liquid agrochemical formulations, such as pesticides, insecticides and herbicides, industrial solvents.

The term “nucleic acid” as used in the present specification is understood to encompass deoxyribonucleic acid (DNA) or ribonucleic acid (RNA). Besides the usual nucleobases or ribonucleosides adenine/adenosine (A), thymine/thymidine (T), cytosine/cytidine (C), guanine/guanosine (G) and uracil/uridine (U), the nucleic acid can include nucleobase and/or ribonucleoside analogs, such as inosine. At its 5′- and/or 3′ end, the nucleic acid can have modifications which increase its stability. The length of the nucleic acids used to mark products is preferably 10 to 5,000 bp, 15 to 4,500 bp, 20 to 4,000 bp, 25 to 2,000 bp, 30 to 1,500 bp, 35 to 1,000 bp, 40 to 500 bp, 45 to 250 bp, 50 to 200 bp, 55 to 100 bp. The nucleic acid used for marking may be a linear or circular nucleic acid. If it is circular, the nucleic acid may be a plasmid or cosmid. The nucleic acid used for marking may also be packaged. For example, the nucleic acid used for marking may be present in a protein coat, for example as a phage. The nucleic acid may also be packaged in a liposome. Besides the sequence which codes the desired character sequence (referred to herein as the “coding region”), the nucleic acid used to mark a product in accordance with the invention may comprise other regions with sequences which enable or facilitate the amplification and/or sequencing of the coding region. The nucleic acid used to mark a product may comprise as further regions at least two primer hybridizing sites of which at least one is located in the 5′ direction (upstream) of the coding region and at least one in the 3′ direction (downstream) of the coding region. The primer hybridizing sites represent regions of the nucleic acid with a sequence which is complementary to the particular sequence of the primers (oligonucleotides) used for amplification (duplication) or sequencing of the nucleic acid and which thus allows binding (hybridization) of the primers to the nucleic acid at the primer hybridizing sites. The length of the primer hybridizing sites corresponds to the length of the primers used. The primer hybridizing sites may directly adjoin the coding region of the nucleic acid or may be separated therefrom by spacers. The spacers preferably have a length of 10 to 200 bp, 20 to 150 bp, 30 to 100 bp, 50 to 80 bp. The nucleic acid sequence in the particular spacer regions may be any sequence or a defined sequence, for example a signal sequence. The nucleic acids used for marking products preferably comprise two spacers which separate the primer hybridizing sites in the 5′ and 3′ direction from the coding region by a distance of preferably 10 to 100 bp, 15 to 90 bp,20 to 80 bp,20 to 70 bp, 25 to 50 bp.

The term “character” as used in the present specification is understood to encompass digits, letters or symbols. The juxtaposition of characters in any order is referred to herein as a character sequence. A character sequence is, for example, a juxtaposition of digits or letters. A character sequence can also be a combination of at least one digit or figure and at least one letter or a juxtaposition of letters. A character sequence is preferably an alpha-numeric character sequence which consists of a combination of letters and figures and to which a meaning, for example a product or batch number, is attributed.

The term “PCR” or “polymerase chain reaction” as used in the present specification denotes an enzyme- and primer-based DNA amplification. The primers (oligonucleotides) used bind specifically to primer hybridizing sites which flank the coding region on its 5′ or 3′ side.

The terms “quantitative PCR”, “real-time PCR” or “real-time polymerase chain reaction” as used in the present specification denote an enzyme- and primer-based DNA amplification which allows quantification (quantity determination) of the nucleic acid present in a sample (Isabel Taverniers et al. 2001).

The terms “RT-PCR” or “Reverse Transcriptase PCR” or “RT polymerase chain reaction” denote a process for enzyme- and primer-based RNA amplification. To this end, a certain messenger RNA (mRNA) is first transcribed into complementary DNA (cDNA) by a reverse transcriptase. This is followed by a PCR or quantitative PCR.

The term “primer” as used in the present specification denotes single-stranded DNA oligonucleotides which hybridize specifically onto a single-stranded DNA or RNA matrix to form a DNA double helix or DNA-RNA double helix and which have a free 3′-OH end. This molecular primer matrix structure is suitable as the starting point for a DNA- or RNA-dependent DNA polymerase. The primers used in the PCR have a length of 6-50 bases, sequence-specific PCR generally requiring primers with lengths upwards of ca. 15 bases. Preferred primer lengths are 6-50, 10-45, 12-40, 15-35, 15-30, 20-45, 25-40 bases.

One aspect of the present invention relates to a process for marking a product with one or more nucleic acids, the process comprising the step of bringing a product to be marked into contact with a nucleic acid, the nucleic acid having a nucleic acid sequence to which a character sequence is assigned.

The nucleic acid used for marking a product may be associated with the product in various ways. The nucleic acid may be present throughout the product or in a part or parts of the product. The nucleic acid may be uniformly distributed throughout the product or in only a part of the product.

In one embodiment of the present invention, the nucleic acid is contacted with the product to be marked by directly adding the nucleic acid to the product. If the product is a fluid, a liquid or a semisolid product or a powder, the nucleic acid is directly mixed with the product. If the product to be marked is a solid which is liquid during the production process, the nucleic acid is added to the product during the liquid phase of the product and is present in the solid product after the solidification process. Alternatively, the nucleic acid may be applied to the surface of the product. Processes for applying the nucleic acid to a solid product include color coating, spray coating, brush coating, vapor deposition, dip coating or mechanical application. Alternatively, the nucleic acid may be introduced into coating compositions which are subsequently applied to the surface of the product to be marked. Examples of coating compositions include paints, lacquers, plastic- or rubber-based coatings and other coatings known from the prior art. These coatings may be applied, for example, to credit cards, holograms, product packaging, labels or other visual markings (for example trade marks or logos). The nucleic-acid-containing coatings may be directly applied to the surface of the product to be marked.

In another embodiment of the invention, the nucleic acid is dissolved or suspended in an ink formulation and applied to the product by, for example, an ink jet process.

In one embodiment of the present invention, the nucleic acid used for marking the product is present in a concentration of preferably 10⁴ to 10¹⁰ copies of DNA or RNA per gram of product, more preferably 10⁴ to 10⁸ copies of DNA or RNA per gram of product and most preferably 10⁴ to 10⁶ copies of DNA or RNA per gram of product.

According to the present invention, the nucleic acid used for marking a product has a nucleic acid sequence which codes a character sequence, for example an alpha-numeric letter and/or digit sequence (number).

The coding of a number, particularly a decimal number, is generally done by converting the decimal number to be coded into the binary (2 digit), tertiary (3 digit) or quaternary (4 digit) number system. Identical digits of the resulting numbers in the various number systems are then each assigned to a nucleobase or a nucleotide. The coding region of the nucleic acid used for marking the product is then synthesized, the nucleotide sequence corresponding to the digit sequence of the binary, tertiary or quaternary system number.

The number obtained after conversion of the decimal number consists of the digits 0 and 1 in the case of the binary system, of the digits 0, 1 and 2 in the case of the tertiary system and of the digits 0, 1, 2 and 3 in the case of the quaternary system. For coding, a nucleotide used in the nucleotide sequence is assigned to a digit. If the conversion is into the binary system, the digit 0 can be assigned, for example, to A, T, C or G. The digit 1 may also be assigned to one of the nucleotides A, T, C or G (it is important to assign both digits to different nucleotides). Where the tertiary and quaternary system is used, the digits are correspondingly assigned. In the case of the tertiary system, this means that the digits 0, 1 and 2 are each assigned to one of the nucleotides A, T, C or G. In the case of the quaternary system, all 4 nucleotides A, T, G and C are assigned to a digit of 0, 1, 2 and 3. With all the number systems used, the assignment is random. If, in addition to the nucleotides A, T, G and C, other nucleotide analogs are used in the nucleotide sequence, for example inosine, the decimal number may be converted into the quinternary (5 digit) number system or each correspondingly higher number system depending on the number of different nucleotides present in the nucleotide sequence.

In a preferred embodiment, the decimal number to be coded, for example the product and/or batch number, may be converted into the binary system. In this case, the digits 0 and 1 each correspond to one of the nucleotides A, T, G or C where the nucleic acid is DNA. Where the nucleic acid is RNA, the digits 0 and 1 each correspond to one of the nucleotides A, U, G or C.

In another preferred embodiment, the digit 0 is assigned to the nucleotide A and the digit 1 to the nucleotide G. If, for example, the number 33380220 is converted into the binary number code, the digit sequence will be 1111111010101011101111100. If the nucleotide A is assigned to the digit 0 and the nucleotide G to the digit 1, the result is the DNA sequence corresponding to the above-mentioned digit sequence, GGGGGGGAGAAGAGGGAGGGGAA.

The coding of letters of an alpha-numeric character sequence by means of the sequence of the coding region of the nucleic acid used for marking a product is carried out according to which system was used for converting the number part of the alpha-numeric character sequence. The nucleotides used for coding the letters are not those used for coding the number part and are therefore freely available. If the number part was converted into the binary system and if the coding nucleic acid is an unmodified DNA molecule (i.e. was synthesized without using nucleotide analogs), the two remaining nucleotides are available for coding letters. If the digits 0 and 1 are taken, for example, by the nucleotides A and G, the remaining nucleotides C and T are available for coding letters. Depending on the number of different letters required for coding, the letters are coded by triplets, quadruplets or quintuplets. A triplet is a sequence of 3 nucleotides, a quadruplet is a sequence of 4 nucleotides and a quintuplet is a sequence of 5 nucleotides which, as a unit, code a character, for example a letter. Where triplets are used and where two free nucleotides are available for coding, at most 8 (=2³) letters or characters can be coded; where a quadruplet is used, at most 16 (=2⁴) letters or characters can be coded and, where a quintuplet is used, at most 32 (=2⁵) letters or characters can be coded.

In a preferred embodiment of the present invention, the coding of letters using the nucleotides C and T available for coding is carried out by a quintuplet code because all 26 letters of the German alphabet can be coded with a quintuplet code. The letters A to Z of the German alphabet are preferably coded by the following quintuplets:

| | | | | | |
|---|---|---|---|---|---|
| A | CCCCC | B | CCCCT | C | CCCTC |
| D | CCTCC | E | CTCCC | F | TCCCG |
| G | CCCTT | H | CCTTC | I | CTCTC |
| J | TCCTC | K | CCTCT | L | CTTCC |
| M | TCTCC | N | TCCCT | O | TTCCT |
| P | TTCTC | Q | TTTCC | R | CTTTC |
| S | CCTTT | T | CTTTT | U | TTTTT |
| V | TTCCC | W | TCTTC | X | TCCTT |
| Y | CTCTT | Z | TTCCC | | |

The expert understands that, depending on the number of characters to be coded, other quintuplet codings can be formed and assigned to the particular characters. The expert also understands that, for coding the individual letters or characters, the individual nucleotides of the quintuplet can be permutated in any other way, so that a unique quintuplet is assigned to each letter or character.

After assigning the individual nucleotides to the individual digits of the resulting binary, tertiary or quaternary system number or the corresponding triplets, quadruplets or quintuplets to the letters/characters of the alpha-numeric character sequence to be coded, the coding nucleotide sequence is compiled and the nucleic acid molecules with the coding nucleotide sequence, i.e. the coding region, are produced. The coding region is preferably flanked at its 5′ and 3′ end by sequences which are complementary to the nucleotide sequence of primers (primer hybridizing sites) used for the amplification and/or sequencing of the coding region lying between the primers. The primer hybridizing sites are preferably separated from the coding region by spacers. The production of the nucleic acids encompassing the coding region and, optionally, primer hybridizing sites and/or spacers is preferably carried out by in vitro synthesis. The synthesized nucleic acid can then be inserted into a vector, for example a plasmid, by standard molecular biological methods.

In another preferred embodiment, characters, numbers/digits and letters are coded using nucleotide triplets. In this case, one group of three nucleotides represents a character, a number/digit or a letter. If the nucleic acid according to the invention consists of the nucleotides A, T, C and G, it is possible using triplets to code at most 64 (=4³) characters, numbers and/or letters. The characters, numbers and/or letters can be assigned as required to the particular triplets. For example, numbers/digits, letters and characters can be assigned as shown in the following:

| | | | | |
|---|---|---|---|---|
| caa = 0 | cag = 1 | cac = 2 | cat = 3 | cca = 4 |
| ccg = 5 | cct = 6 | cga = 7 | cgc = 8 | cgg = 9 |
| gaa = a | gac = b | gag = c | gat = d | gca = e |
| gcc = f | gcg = g | gct = h | gga = i | ggc = j |
| ggt = k | gta = l | gtc = m | gtg = n | gtt = o |
| taa = p | tac = q | tag = r | tat = s | tca = t |
| tcc = u | tcg = v | tct = w | tga = x | tgc = y |
| tgt = z | | | | |
| ata = . | atc = , | atg = - | att = ; | ggg = [void] |

The expert understands that, depending on the number of characters to be coded, other and/or further triplets can be formed and assigned to the particular characters or to further characters.

According to the invention, the process according to the invention additionally comprises a step for detecting the nucleic acid used to mark the product and a step for determining the sequence of the nucleic acid, more particularly the sequence of the coding region. Using the process according to the invention, the nucleic acid in or on the product can be detected by examining the entire product, a part of the product or an extract of the product. The nucleic acid can be isolated by any of the methods known from the prior art for isolating nucleic acids. If the nucleic acid used for marking the product is plasmid DNA, the DNA can be isolated using the CTAB/Wizard isolating process.

In a specific embodiment of the present invention, the sequence of nucleic acid isolated from the product is determined. If only small quantities of the nucleic acid are used for marking the product, the isolated nucleic acid is amplified before determination of the sequence. The nucleic acid is amplified by known methods for amplifying nucleic acids.

The isolated nucleic acid is amplified in order to overstep the sensitivity threshold of a following physicochemical analysis system (principle of selective signal amplification). The most commonly used DNA amplification technique is the polymerase chain reaction (PCR) (U.S. Pat. No. 4,683,195). In this enzyme-chemical process, the nucleic acids are selectively duplicated using heat-stable DNA-dependent DNA polymerases and PCR primers. The primers used in accordance with the present invention are complementary to sequences which flank the sequence coding the alpha-numeric character sequence, i.e. the coding region of the nucleic acid, on its 5′ and 3′ side. By combining two PCR primers to form a PCR primer pair, of which the individual primers are each capable of binding to a matrix strand of the DNA target sequence and offset in the 3′ direction in relation to their orientations, the DNA region lying in between is exponentially amplified. PCR is a cyclic process, each cycle consisting of at least two temperature steps, the melting of the double-stranded DNA at ca. 90-95° C. and the addition and lengthening of the primers at ca. 50-75° C. Thermocyclers are generally used for this purpose.

If the nucleic acid used for marking the product is RNA, it is transcribed into cDNA by a reverse transcription before the amplification step. In this case, the reverse transcription of the RN and subsequent cDNA amplification are preferably carried out by an RT-PCT.

The sequence of the nucleic acid isolated from the product and amplified is determined by sequencing. This sequencing is carried out by known methods. The nucleic acid sequencing methods of Maxam and Gilbert or Sanger (Sambrook et al. 1989) are mentioned by way of example.

The nucleotide sequence of the coding region of the nucleic acid thus determined is decoded in order to determine the alpha-numeric product and/or batch number of the product. Decoding is carried out by first dividing the nucleotide sequence determined into the individual triplets, quadruplets or quintuplets and assigning them to the predetermined letters or characters. In the same way, the individual nucleotides are assigned in order to the particular predetermined characters of the number system used. Finally, the number obtained is converted into the corresponding number of the decimal system. For decoding, it is necessary to know which nucleotide of the coding region is the first nucleotide that is part of a coding unit, for example a triplet, quadruplet or quintuplet, or which nucleotide represents the first digit of the coded binary, tertiary or quaternary system. To this end, one or both of the spacers present in the nucleic acid can have at least one signal nucleotide sequence which is situated at a defined distance from the first coding unit (for example triplet, quadruplet or quintuplet or individual nucleotide) in the coding region. For example, the signal nucleotide sequence can be situated in the spacer which flanks the coding region in the 5′ direction. The signal nucleotide sequence can be, for example, a sequence of GGGGGGGG (octa-G sequence) which indicates that the first nucleotide which codes a digit or is part of a coding unit (for example a triplet, quadruplet or quintuplet) is situated at a defined distance from the eighth guanosine unit, for example 20 nucleotides, in its 3′ direction.

In another embodiment of the present invention, the quantity of nucleic acid present in the marked product is determined (nucleic acid quantification) in order to determine whether the product of the honest manufacturer has been blended, i.e. diluted. Examples of DNA quantification processes include UV spectrometry at 260 nm, fluorometry using fluorophores which bind specifically to DNA, densitometric processes using DNA reference fragments and quantitative real-time PCR (qPCR). The quantity of nucleic acid present in the product marked in accordance with the invention is preferably determined by quantitative real-time PCR.

In recent years, PCR technology has made major advances through the development of fast thermocyclers and the establishment of fluorescence-based determination of the double-stranded DNA fragments generated after each amplification cycle which allows quantification by “rapid-cycle real-time PCR” analyses (for example LightCycler®, Roche Diagnostics Corp.; ABI Prism® 7000 SDS, Applied Biosystems, Darmstadt, Germany). The sensitive quantification is based on the detection of increasing fluorescence during the exponential phase of PCR in relation to the quantity of DNA originally used for the reaction. The quantification is based in particular on determination of the “cycle threshold” (ct) value, the first PCR reaction round with detectable fluorescence. With the assistance of external DNA standards of known concentration, an absolute quantification can be achieved. In general, the fluorescence can be detected sequence-specifically by hybridization probes or TaqMan® probes (based on the 5′→3′ exonuclease activity of the Taq DNA polymerase; Roche Diagnostics and Applied Biosystems, Darmstadt, Germany) or non-sequence-specifically using dyes which bind selectively to double-stranded DNA (more particularly SybrGreen, Invitrogen Inc.).

Determining whether the product of the honest manufacturer has been diluted presupposes that each product or each product batch contains a predetermined quantity of the nucleic acid used for marking. The nucleic acid is preferably present in a quantity of 10⁴ to 10¹⁰ copies DNA or RNA per gram of product, more preferably 10⁴ to 10⁸ copies DNA or RNA per gram of product and most preferably 10⁴ to 10⁶ copies DNA or RNA per gram of product. The predetermined quantity of nucleic acid added (per gram of product) with each product or product batch is defined as 100% and characterizes the product as original, i.e. genuine and undiluted, product. At least one “re-set” sample of each product marked in accordance with the invention or each product batch marked in accordance with the invention is taken and stored. After isolation of the DNA from the re-set sample, the quantity of DNA per gram in the re-set sample taken is determined by real-time PCR. The ct-value determined is defined as 100%. To establish a calibration curve, dilutions of the DNA isolated from the re-set sample (for example a 10%, 20%, 30%, 40%, 50%, 60%, 70% or 80% dilution) are prepared and the respective ct-values are determined by real-time PCR. The testing of a product for authenticity or undilution is carried out by isolating the nucleic acid, i.e. the DNA, used for marking from the product and determining the quantity of DNA per gram of the product to be tested by real-time PCR. The ct-value determined for the product to be tested is compared with the calibration ct values determined from the dilutions of the DNA obtained from the corresponding re-set sample. If, for example, a ct-value of 31 was determined for the 50% DNA dilution of the re-set sample and if the DNA quantification of the corresponding product to be tested for authenticity also produces a ct-value of 31, it may be concluded that the tested product has been 50% blended, i.e. diluted, with other materials.

The present invention also relates to the use of a nucleic acid coding a character sequence, for example a product or batch number, for the falsification-proof marking of a product.

The present invention also relates to a product marked with nucleic acids with a nucleic acid sequence which codes a character sequence, for example a word or letter sequence.

### EXAMPLE

The character sequence (here: word or letter sequence) “Cognis Original—We Know How” was coded into a DNA sequence using the following triplet code:

| | | | | |
|---|---|---|---|---|
| gaa = a | gac = b | gag = c | gat = d | gca = e |
| gcc = f | gcg = g | gct = h | gga = i | ggc = j |
| ggt = k | gta = l | gtc = m | gtg = n | gtt = o |
| taa = p | tac = q | tag = r | tat = s | tca = t |
| tcc = u | tcg = v | tct = w | tga = x | tgc = y |
| tgt = z | | | | |
| ata = . | atc = , | atg = - | att = ; | |

Each letter or each character of the above character sequence corresponds to a specific triplet. For the triplet code shown above, the character sequence “Cognis Original—We Know How” corresponds to the nucleotide sequence 5′-gaggttgcggtgggatatgtttagggagcgggagtggaagtaatgtctgcaggtgtggtttctgctgtttct-3′ (SEQ ID NO:1). A nucleic acid molecule having that nucleotide sequence was synthesized by an outside service laboratory. Using standard molecular-genetic techniques (Sambrook et al. (1989)), the synthesized nucleic acid molecule was inserted as a coding region into the multiple cloning site of the plasmid pUC19 by conventional cloning processes. The plasmid pUC19-Cognis thus generated was added to various matrices. A PIT emulsion (Emulgade CM SE-PF), a spreading oil (Cetiol OE) and an anionic surfactant (Texapon N70) are mentioned by way of example in this regard. The matrices had been obtained from Cognis Deutschland GmbH & Co. KG (Germany). The quantities of nucleic acid added to the particular matrices in various test batches were 10³, 10⁴, 10⁵ and 10⁶ plasmid molecules/g matrix. The plasmids were uniformly distributed in the matrices and then isolated from the matrices again by the CTBA Wizard DNA isolation process (http://gmocrl.jrc.it/summaries/TC1507-DNAextrc.pdf). Using standard PCR Sambrook et al. (1989)), the nucleic acid molecules thus isolated were amplified by the primers Tn5-1: 5′-GGA TCT CCT GTC ATC T-3′ (SEQ ID NO: 2) and pep3-6: 5′-CAC TCT TGT CTC TTG TCC TC-3′ (SEQ ID NO: 3). With all the tested matrices, a specific DNA amplificate was obtained with a plasmid quantity originally used for marking of 10⁴ plasmid molecules per gram matrix upwards. After the integrity of the nucleic acid pUC19-Cognis used for marking the matrices had been confirmed by the DNA amplification, the specific DNA amplificate encompassing the coding region was sequenced using standard molecular-genetic processes (Sambrook et al. (1989)). The determined nucleotide sequence of the coding region present in the multiple cloning site of the PUC vector was 5′-gaggttgcggtgggatatgtttagggagcgggagtggaagtaatgtctgcaggtgtggtttctgctgtttct-3′. This is identical with the sequence of the synthesized coding region (see SEQ ID NO:1). By assigning the individual triplets to the corresponding characters, the DNA sequence was decoded and produced the original character sequence “Cognis Original—We Know How”.

### Literature

Isabel Taverniers, Pieter Windels, Erik Van Bockstaele, Marc De Loose (2001). Use of cloned DNA fragments for event-specific quantification of genetically modified organisms in pure and mixed food products, Eur. Food Res. Technol. 213:417-424
Sambrook J, Fritsch E F, Maniatis T (1989). Molecular cloning. A laboratory manual. 2^{nd} Edition, Cold Spring Harbor Laboratory Press, Cold Spring Harbor, USA.
WO 87/06383
WO 95/06249
WO 98/33162
EP 1 199 371A2
EP 0 409 842 B1
EP 0 327 163 B1
U.S. Pat. No. 4,683,195

1. A process for marking a product to prove its identity and origin, the process comprising the step of marking the product with a nucleic acid, characterized in that the nucleic acid has a coding region of which the sequence is associated with a sequence of characters.

2. A process as claimed in claim 1, characterized in that the sequence of characters is a sequence of letters and/or digits.

3. A process as claimed in claim 2, characterized in that the digit sequence is a decimal number.

4. A process as claimed in claim 3, characterized in that the coding of the decimal number in the sequence of the coding region is carried out by converting the decimal number into another number system and assigning identical digits of the resulting number of the other number system to a nucleotide of the nucleic acid.

5. A process as claimed in claim 4, characterized in that the decimal number is converted into the binary, tertiary or quaternary number system.

6. A process as claimed in claim 2, characterized in that the letters are coded by assigning a unique triplet, quadruplet or quintuplet of nucleotides of the nucleic acid to be synthesized to each letter.

7. A process as claimed in claim 1, characterized in that a triplet of nucleotides in the coding region of the nucleic acid is assigned to each character of the character sequence.

8. A process as claimed in claim 1, characterized in that the nucleic acid further comprises primer hybridizing sites and/or spacers.

9. A process as claimed in claim 1, characterized in that the nucleic acid is a DNA or RNA.

10. A process as claimed in claim 1, characterized in that the character sequence denotes the product number, the batch number, the time of manufacture and/or the place of manufacture of the product.

11. A process for determining the identity and origin of a product marked by the process claimed in claim 1, characterized in that it comprises the following steps:
a) isolating the nucleic acid used for marking from the product,
b) amplifying the isolated nucleic acid,
c) determining the sequence of the nucleic acid and
d) decoding the nucleic acid sequence of the coding region of the nucleic acid.

12. A process as claimed in claim 11, characterized in that the nucleic acid is amplified by PCR.

13. A process as claimed in claim 11, characterized in that decoding is carried out by assigning each nucleotide of the coding region of the nucleic acid to the previously determined digit of the number system used and converting the resulting number of the number system used into the corresponding decimal number.

14. A process as claimed in claim 11, characterized in that decoding is carried out by assigning each unique triplet, quadruplet or quintuplet of nucleotides of the coding region of the nucleic acid to the letter determined beforehand.

15. A process as claimed in claim 11, characterized in that decoding is carried out by assigning each unique triplet of nucleotides of the coding region of the nucleic acid to the character determined beforehand.


SEQUENCE LISTING (3 sequences)

SEQ ID NO: 1 - DNA, 72 bp, Artificial Sequence
  Sequence of the coding region in pUC-Cognis
gaggttgcggtgggatatgtttagggagcgggagtggaagtaatgtctgcaggtgtggtttctgctgtttct

SEQ ID NO: 2 - DNA, 16 bp, Artificial Sequence
  Primer Tn5-1
ggatctcctgtcatct

SEQ ID NO: 3 - DNA, 20 bp, Artificial Sequence
  Primer pep3-6
cactcttgtctcttgtcctc